(19) United States
(12) Patent Application Publication
(10) Pub. No.: US 2009/0175831 A1
(43) Pub. Date: Jul. 9, 2009

(54) ADAPTABLE MESSENGER RIBONUCLEIC ACID MEDICAL TREATMENT DEVICE TO MANAGE DIABETES MELLITUS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 11/969,887

(22) Filed: Jan. 5, 2008

(51) Int. Cl. A61K 35/76; A61P 3/10

(52) U.S. Cl. 424/93.6

(57) ABSTRACT

Diabetes mellitus is a disease of elevated blood glucose, often directly related to a deficiency in insulin production or insulin receptor production. The innovative strategy of treatment described here utilizes modified viruses to act as a transport vehicle to deliver to target cells in the body, messenger RNA molecules. Delivering to the beta cells in the body the messenger RNA molecules needed to construct insulin or insulin receptors will lead to enhanced production of biologically active insulin or insulin receptors by beta cells as necessary, which will lead to correcting deficiencies in insulin or insulin receptors the result of which will help properly regulate blood glucose levels throughout the body utilizing innate regulatory mechanisms.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to correct a protein deficiency in the body by increasing the intracellular production of the deficient protein by utilizing a modified virus to insert one or more messenger ribonucleic acid molecules into one or more cells of the body.

2. Description of Background Art

Diabetes mellitus represents an important health issue that affects a significant portion of the world population. In the United States, about 16 million people suffer from diabetes mellitus. Every year, about 650,000 additional people are diagnosed with the disease. Diabetes mellitus is the seventh leading cause of all deaths.

Diabetes mellitus represents a state of hyperglycemia, a serum blood sugar that is higher than what is considered the normal range for humans. Glucose, a six-carbon molecule, is a form of sugar. Glucose is absorbed by the cells of the body and converted to energy by the processes of glycolysis, the Krebs cycle and phosporylation. Insulin, a protein, facilitates the absorption of glucose into cells. Normal range for blood sugar in humans is generally defined as a fasting blood plasma glucose level of between 70 to 110 mg/dl. For descriptive purposes, the term ‘plasma’ refers to the fluid portion of blood. Diabetes mellitus is classified as Type One and Type Two. Type One diabetes mellitus is insulin dependent, which refers to the condition where there is a lack of sufficient insulin circulating in the blood stream and insulin must be provided to the body in order to properly regulate the blood glucose level. When insulin is required to regulate blood glucose level in the body, this condition is often referred to as insulin dependent diabetes mellitus (IDDM). Type Two diabetes mellitus is noninsulin dependent, often referred to as noninsulin dependent diabetes mellitus (NIDDM), meaning the blood glucose level can be managed without insulin, but by means of diet, exercise or intervention with oral medications. Type Two diabetes mellitus is considered a progressive disease, the underlying pathogenic mechanisms including pancreatic beta cell (also often designated as P-Cell) dysfunction and insulin resistance.

The pancreas serves as an endocrine gland and an exocrine gland. Functioning as an endocrine gland the pancreas produces and secretes hormones including insulin and glucagon. Insulin acts to reduce levels of glucose circulating in the blood. Beta cells secrete insulin into the blood when a higher than normal level of glucose is detected in the serum. For purposes of this description the terms ‘blood’, ‘blood stream’ and ‘serum’ refer to the same substance. Glucagon acts to stimulate an increase in glucose circulating in the blood. Beta cells in the pancreas secrete glucagon when a low level of glucose is detected in the serum.

Glucose enters the body and then the blood stream as a result of the digestion of food. The beta cells of the Islets of Langerhans continuously sense the level of glucose in the blood and respond to elevated levels of blood glucose by secreting insulin into the blood. Beta cells produce the protein ‘insulin’ in the endoplasmic reticulum and store the insulin in vacuoles until it is needed. When beta cells detect an increase in the glucose level in the blood, beta cells release insulin into the blood from the said storage vacuoles.

Insulin is a protein. An insulin protein consists of two chains of amino acids, an alpha chain and a beta chain, linked by two disulfide (S—S) bridges. The alpha chain consists of 21 amino acids. The beta chain consists of 30 amino acids.

Insulin interacts with the cells of the body by means of a cell-surface receptor termed the ‘insulin receptor’ located on the exterior of a cell's ‘outer membrane’, otherwise known as the ‘plasma membrane’. Insulin interacts with muscle and liver cells by means of the insulin receptor to rapidly remove excess blood sugar when the glucose level in the blood is higher than the upper limit of the normal physiologic range. Recognized functions of insulin include stimulating cells to take up glucose from the blood and convert it to glycogen to facilitate the cells in the body to utilize glucose to generate biochemically usable energy, and to stimulate fat cells to take up glucose and synthesize fat.

Diabetes Mellitus may be the result of one or more factors. Causes of diabetes mellitus may include: (1) mutation of the insulin gene itself causing miscoding, which results in the production of ineffective insulin molecules; (2) mutations to genes that code for the ‘transcription factors’ needed for transcription of the insulin gene in the DNA to create messenger RNAs which facilitates the manufacture of the insulin molecule; (3) mutations of the gene encoding for the insulin receptor, which produces inactive or an insufficient number of insulin receptors; (4) mutation to the gene encoding for glucokinase, the enzyme that phosphorylates glucose in the first step of glycolysis; (5) mutations to the genes encoding portions of the potassium channels in the plasma membrane of the beta cells, preventing proper closure of the channel, thus blocking insulin release; (6) mutations to mitochondrial genes that as a result, decreases the energy available to be used facilitate the release of insulin, therefore reducing insulin secretion; (7) failure of glucose transporters to properly permit the facilitated diffusion of glucose from plasma into the cells of the body.

A eukaryote refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure receptors that the cell uses to detect properties of the environment surrounding the cell membrane. The cytoplasm acts as a filling medium inside the boundaries of the plasma cell membrane and is comprised mainly of water and nutrients such as amino acids, oxygen, and glucose. The nucleus, organelles, and ribosomes are suspended in the cytoplasm. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex. Floating in the cytoplasm, but also located in the endoplasmic reticulum and mitochondria are ribosomes. Ribosomes are protein structures comprised of several strands of proteins that combine and couple to a messenger ribonucleic acid (mRNA) molecule. More than one ribosome may be attached to a single mRNA at a time. Ribosomes decode genetic information in a mRNA molecule and manufacture proteins to the specifications of the instruction code physically present in the mRNA molecule.

The majority of the deoxyfibonucleic acid (DNA) comprises the chromosomes, double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases, to produce ribonucleic acid (RNA). RNA is a single strand of genetic information comprised of coded arrangements of four nucleotides: adenine, uracil, guanine and cytosine. Some types of RNAs are classified as messenger RNAs (mRNA), transport RNAs (tRNA) and ribosomal RNAs (rRNA).

Proteins are comprised of a series of amino acids bonded together in a linear strand, sometimes referred to as a chain; a protein may be further modified to be a structure comprised of one or more similar or differing strands of amino acids bonded together. Insulin is a protein structure comprised of two strands of amino acids, one strand comprised of 21 amino acids long and the second strand comprised of 30 amino acids, the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors. Messenger RNAs (mRNA) are created by transcription of DNA, they exit the nucleus of the cell, and are utilized as protein manufacturing templates by ribosomes. A ribosome is a protein complex that manufactures proteins by deciphering the instruction code located in a mRNA molecule. When a specific protein is needed, pieces of the ribosome complex bind around the strand of a mRNA that carries the specific instruction code that will generate the required protein. The ribosome traverses the mRNA strand and deciphers the genetic information coded into the sequence of nucleotides that comprise the mRNA molecule.

Transport RNAs (tRNA) are constructed in the nucleus or in the mitochondria, and are coded for one of the 20 amino acids the cells of the human body use to construct proteins. Once a tRNA is created by transcription of the DNA, the tRNA seeks out the type of amino acid it has been coded for and attaches to that specific amino acid. The tRNA then delivers the amino acid it carries to a ribosome that is waiting for that specific amino acid. Proteins are manufactured by the ribosomes binding together sequences of amino acids. The order by which the amino acids are bonded together is dictated by the way the mRNA is constructed and how the ribosome interprets the information encoded in the string of nucleotides present in the mRNA strand.

A sequence of three nucleotides present in a mRNA molecule represents a unit of information referred to as a codon. Codons code for all of the 20 amino acids used to construct protein molecules and also for START and STOP commands. In the process known as translation, the ribosome decodes the codons present in the mRNA, initiating the protein manufacturing process at a START codon, then interfacing with tRNAs carrying the amino acids that match the sequence of codons in the mRNA as the ribosome traverses the length of the mRNA molecule. The ribosome functions as a protein factory by taking amino acids delivered by tRNAs and binding the amino acids together in the order dictated by the sequence of codon instructions coded into the mRNA template as directed by the manner of the nucleic acid arrangement in the mRNA molecule. Protein synthesis ceases when a ribosome encounters a STOP code. The protein molecule is released by the ribosome.

The insulin molecule is a protein produced by beta cells located in the pancreas. The ‘insulin messenger RNA’ is created in a cell by transcribing the insulin gene from nuclear DNA in the nucleus of the cell. The native messenger RNA (mRNA) for insulin then travels to the endoplasmic reticulum, where numerous ribosomes engage these mRNA molecules. Many ribosomes may be attached to a single strand of mRNA simultaneously, each generating an identical copy of the protein as dictated by the information encoded in the mRNA. Insulin is produced by ribosomes translating the information in a mRNA molecule coded for the insulin protein, which produce strands of amino acids that are coded for an immature form of the biologically active insulin molecule referred to as ‘pro-insulin’. Once the pro-insulin molecule is generated it then undergoes modification by several enzymes including prohormone convertase one (PC1), prohormone convertase two (PC2) and carboxypeptidase E, which results in the production of a biologically active insulin molecule. Once the biologically active insulin protein is generated it is stored in a vacuole in the beta cell to await being released into the blood stream.

The insulin receptor, prohormone convertase one (PC1), prohormone convertase two (PC2) and carboxypeptidase E are produced in a similar fashion as to how pro-insulin and insulin are produced in a beta cell. A messenger RNA is transcribed from DNA, specific for either the insulin receptor, prohormone convertase one (PC1), prohormone convertase two (PC2) or carboxypeptidase E. When a messenger RNA coded for an insulin receptor is present and available, ribosomes will attach to the mRNA and generate insulin receptor proteins. When a messenger RNA coded for either prohormone convertase one (PC1), prohormone convertase two (PC2) or carboxypeptidase E is present and available, ribosomes will attach to the mRNA, decode the instructions in the mRNA molecule and generate the protein.

Insulin receptors, which appear on the surface of cells, offer binding sites for insulin circulating in the blood. When insulin binds to an insulin receptor, the biologic response inside the cell causes glucose to undergo processing in the cytoplasm. Processed glucose molecules then enter the mitochondria. The mitochondria further process the modified glucose molecules to produce usable energy in the form of adenosine triphosphate molecules (ATP). Thirty-eight ATP molecules may be generated from one molecule of glucose during the process of aerobic respiration. ATP molecules are utilized as an energy source by biologic processes throughout the cell.

The current medical therapeutic approach to the management of diabetes mellitus has produced limited results. Patients with diabetes generally struggle with an inadequate production of insulin, or an ineffective release of biologically active insulin molecules, or a release of an insufficient number of biologically active insulin molecules, or an insufficient production of cell-surface receptors, or a production of ineffective cell-surface receptors, or a production of ineffective insulin molecules that are unable to interact properly with insulin receptors to produce the required biologic effect. Type One diabetes requires administration of exogenous insulin. The traditional approach to Type Two diabetes has generally first been to adjust the diet to limit the caloric intake the individual consumes. Exercise is used as an initial approach to both Type One and Type Two diabetes as a means of up-regulating the utilization of fats and sugar so as to reduce the amount of circulating plasma glucose. When diet and exercise are inadequate in properly managing Type Two diabetes, oral medications are often introduced. The action of sulfonylureas, a commonly prescribed class of oral medication, is to stimulate the beta cells to produce additional insulin receptors and enhance the insulin receptors' response to insulin. Biguanides, another form of oral treatment, inhibit gluconeogenesis, the production of glucose in the liver, thereby attempting to reduce plasma glucose levels. Thiazolidinediones (TZDs) lower blood sugar levels by activating peroxisome proliferator-activated receptor gamma (PPAR-γ), a transcription factor, which when activated regulates the activity of various target genes, particularly ones involved in glucose and lipid metabolism. If diet, exercise and oral medications do not produce a satisfactory control of the level of blood glucose in a diabetic patient, exogenous insulin is injected into the body in an effort to normalize the amount of glucose present in the serum. Insulin, a protein, has not successfully been made available as an oral medication to date due to the fact that proteins in general become degraded when they encounter the acid environment present in the stomach.

Despite strict monitoring of blood glucose and potentially multiple doses of insulin injected throughout the day, many patients with diabetes mellitus still experience devastating adverse effects from elevated blood glucose levels. Microvascular damage and elevated tissue sugar levels contribute to such complications as renal failure, retinopathy involving the eyes, neuropathy, and accelerated heart disease despite aggressive efforts to maintain the blood sugar within the physiologic normal range using exogenous insulin by itself or a combination of exogenous insulin and one or more oral medications. Diabetes remains the number one cause of renal failure in this country. Especially in diabetic patients that are dependent upon administering exogenous insulin into their body, though dosing of the insulin may be four or more times a day and even though this may produce adequate control of the blood glucose level to prevent the clinical symptoms of hyperglycemia; this does not unerringly supplement the body's natural capacity to monitor the blood sugar level minute to minute, twenty-four hours a day, and deliver an immediate response to a rise in blood glucose by the release of insulin from beta cells as required. The deleterious effects of diabetes may still evolve despite strict and persistent control of the glucose level in the blood stream.

The current treatment of diabetes may be augmented by the unique approach to utilizing modified viruses as vehicles to transport biologically active messenger ribonucleic acids (mRNA) coded to facilitate the manufacture of pro-insulin and insulin and the enzymes utilized to modify proinsulin to the biologically active insulin molecule and messenger ribonucleic acids (mRNA) coded to manufacture insulin receptors.

Viruses are obligate parasites. Viruses simply represent a carrier of genetic material and by themselves viruses are unable to replicate or carry on any form of biologic function outside their host cell. Viruses are generally comprised of one or more shells constructed of one or more layers of protein or lipid material, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell provides the virus the proper biochemical machinery for the virus to successfully replicate itself.

Protected by the outer coat or envelope, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate their RNA being converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as mRNA. RNA viruses that bypass the host cell's DNA, cause the cell to in general generate copies of the necessary parts of the virus directly from the virus's RNA genome.

The Hepatitis C virus (HCV) is a positive sense RNA virus, meaning a type of RNA that is capable of bypassing the need for involving the host cell's nucleus by having its RNA genome function as messenger RNA. Hepatitis C infects liver cells. The Hepatitis C genome becomes divided once it gains access to the interior of a liver host cell. Portions of the subdivisions of the Hepatitis C genome directly interact with ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to effect the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus offers a naturally occurring vehicle mechanism to transport and insert medically therapeutic messenger ribonucleic acid (mRNA) molecules into specific targeted cells of the human body. The surface probes present on the Hepatitis C virus's outer protein coat can be modified to seek out specific receptors on specific target cells. Once the modified Hepatitis C virus's probes properly engage the cell-surface receptors on a target cell, the modified Hepatitis C virus would insert into the target cell one or more medically therapeutic mRNAs for the purpose of having the target cell generate proteins to achieve a medical therapeutic response.

Current state of gene therapy generally refers to efforts directed toward inserting an exogenous subunit of DNA into a vehicle such as a virus. The vehicle is intended to insert the exogenous subunit of DNA into a target cell. The exogenous DNA subunit then migrates to the target cell's nucleus. The exogenous DNA subunit then inserts into the native DNA of the cell. This represents a permanent alteration of the cell's nuclear DNA. The nuclear transcription proteins then read the exogenous DNA subunit's nucleotide coding to produce the intended cellular response. The approach described hereunder involves RNA versus DNA. DNA is comprised of the nucleotides adenine, thymine, guanine and cytosine. RNA is composed of the nucleotides adenine, uracil, guanine and cytosine. DNA codes for the manufacture of RNAs, which are composed of nucleotides. RNA codes for the manufacture of proteins, which are composed of amino acids. The virus chosen as the transport vehicle, Hepatitis C virus, is a RNA virus versus a virus that naturally carries a DNA genome.

Beta cells located in the Islets of Langerhans in the pancreas are thought to have at least one unique identifying surface receptor. The exterior receptor GPR40 appears specific to beta cells located in the Islets of Langerhans in the pancreas. A virus equipped with a surface probe designed to engage the GPR40 beta cell receptor, could travel the blood stream of the body until it locates a GPR40 receptor on a beta cell, engage the receptor with its surface probe, and then insert the genetic payload it carries into the beta cell. A genetic payload such as one or more messenger RNAs could be used to enhance proper protein production by cells deficient in a particular protein. Hormones are proteins that circulate the body and stimulate biologic activity specific to the hormone's role. In the case of a deficiency of a hormone, production of a deficient hormone could be enhanced by inserting one or more messenger RNAs into specific target cells in the body to stimulate production of the required hormone. In the case of diabetes mellitus, utilizing a modified Hepatitis C virus as a vehicle, messenger RNA molecules could be inserted into beta cells, coded for any protein the individual's beta cells are deficient in producing, with the intention of generating an adequate insulin production and adequate release of insulin into the blood to meet the body's needs.

The utilization of positive sense messenger RNA, does not permanently alter the cell's DNA. Messenger RNAs degrade and become unusable after a time. Use of RNA as a therapeutic modality offers a therapeutic opportunity that could have a reversible or an attenuatable effect when required. Messenger RNA also bypasses the action of decoding the DNA and errors or deficiencies that might occur during transcription. By employing a medically therapeutic virus to carry messenger RNA to cells, deficiencies of any of the approximately 30,000 proteins that comprise the tissues that exist in the body and on the surface of the body can be successfully treated or averted.

### BRIEF SUMMARY OF THE INVENTION

A modified virus is used as a transport medium to carry a payload of one or more messenger ribonucleic acid (RNA) molecules. The modified virus makes contact with a target beta cell located in the Islets of Langerhans in the pancreas by means of the modified virus's exterior probes including one or more probes meant to engage GPR40 exterior cell-surface receptors on a beta cell. Once the virus's exterior probes engage the target cell's receptors, the modified virus inserts into the target cell one or more messenger ribonucleic acid (mRNA) molecules it is carrying. Messenger RNA molecules inserted into the cell act as native messenger RNA molecules and either interact with the cell's ribosomes in the process of protein synthesis or interact with the cell's native enzymes and undergo further modification until the delivered messenger RNA molecule is capable of interacting with the cell's ribosomes in the process of protein synthesis. Medical disease states such as diabetes mellitus that are the result of a deficiency of one or more proteins can be successfully treated by utilizing viruses to insert the proper messenger RNA molecules, into specific cells to enhance the production of proteins that are identified as being deficient, thus correcting the deficiency. The deficiency of insulin production is a prime example of a medical condition that is capable of being corrected by modifying a virus to transport messenger RNA molecules coded for the pro-insulin molecule, the insulin molecule, the insulin receptor molecule, prohormone convertase one (PC1), prohormone convertase two (PC2), and/or carboxypeptidase E, delivering such messenger RNAs to beta cells for the purpose of enhancing the beta cells' production of the insulin molecule and/or the insulin receptor.

### DETAILED DESCRIPTION

Diabetes mellitus is a medical condition often recognized when an individual's fasting blood glucose level is persistently higher than the generally accepted normal range of 60-110 mg/dl. An elevated blood glucose level may occur as the result of a lack of sufficient insulin; a lack of sufficient biologically effective insulin; a deficiency of the number of insulin receptors available to interact with insulin; a deficiency in the number of biologically active insulin receptors available to properly interact with insulin; insufficient release of insulin into the blood stream.

Insulin, a protein, is generated in beta cells located in the Islets of Langerhans in the pancreas. Insulin is produced by decoding DNA through a process called transcription. Initially, transcription of the DNA produces a messenger ribonucleic acid (mRNA) molecule coded for the pro-insulin molecule. This mRNA coded for the ‘pro-insulin’ molecule, is then decoded by one or more ribosomes through a process called translation to produce a chain of amino acids that is referred to as the ‘pro-insulin’ molecule. The ‘pro-insulin’ molecule is modified by enzymes to produce the biologically active ‘insulin’ protein. Insulin molecules are stored in vacuoles in the beta cells of the pancreas. Insulin is released from storage vacuoles in response to a rise in the level of glucose in the blood. Other proteins are manufactured in a similar fashion as pro-insulin and insulin.

Errors in the DNA or errors that occur in the process that generates the messenger RNA or a deficiency in the number of messenger RNA or a deficiency in the number of biologically active messenger RNA results in a deficiency of, or errors in the ‘pro-insulin’ molecule. Deficiencies in the biologically active enzymes intended to modify the ‘pro-insulin’ molecule to produce the biologically active insulin protein may result in deficiencies in adequate insulin production.

Correcting deficiencies or errors associated with the production of the protein insulin would correct diabetes mellitus, when diabetes mellitus is related to an insufficient quantity of biologically active insulin.

The Hepatitis C virus (HCV) is comprised of an outer lipoprotein envelope and an internal nucleocapsid. The virus's genetic payload is carried within the nucleocapsid. The HCV's naturally occurring genetic payload consists of a single molecule of linear positive sense, single stranded RNA approximately 9600 nucleotides in length, which includes: the structural proteins of C, E1, E2, the nonstructural proteins NS1, NS2, NS3, NS4A, NS4B, NS5A, NS5B, p7 and ARFP/F protein. Present on the surface of the outer envelope of the Hepatitis C virus are probes that detect receptors present on the surface of liver cells. The glycoproteins E1 and E2 have been identified to be affixed to the surface of HCV. Portions of the Hepatitis C virus genome, when separated into individual pieces, behave like messenger RNA. Naturally occurring HCV is constructed with surface probes fashioned to recognize a receptor on the surface of a liver cell. Once the naturally occurring HCV's surface probe E2 engages a liver cell's CD81 receptor, and cofactors on the surface of HCV's exterior envelope engage the low density lipoprotein receptor (LDLR) or the scavenger receptor class B type I (SR-BI) on the liver cell, HCV then has the opportunity to insert its RNA genetic payload into the engaged target liver cell.

Replicating viruses and constructing viruses to carry DNA payloads is a form of manufacturing technology that has already been well established and is in use facilitating gene therapy. Replicating viruses and designing these viruses to carry messenger ribonucleic acid as the genetic payload would incorporate similar techniques as already proven useful in current gene therapy technologies.

To carry out the process to manufacture a modified medically therapeutic Hepatitis C virus, messenger RNA would be inserted into the host that would code for the general physical outer structures of the Hepatitis C virus. Messenger RNA would be inserted into the host that would generate surface probes that would target the surface receptors on beta cells. Messenger RNA would be inserted into the host that would generate copies of the messenger RNA that would provide a therapeutic action that would take the place of the Hepatitis C virus's innate genome. Therapeutic messenger RNA that would act as the modified HCV's genome would encode for proteins that would include the pro-insulin molecule, the insulin molecule, the insulin receptor, the enzyme prohormone convertase one, the enzyme prohormone convertase two, the enzyme carboxypeptidase E. Similar to how copies of a naturally occurring Hepatitis C virus is produced, assembled and released from a host cell, copies of the modified medically therapeutic Hepatitis C virus would be produced, assembled and released from a host cell.

To treat the various different forms of diabetes mellitus various combinations of messenger RNA would be inserted into the host, and the host would produce copies of modified Hepatitis C virus that target beta cells and carry a genetic payload consisting of messenger RNA molecules that would consist of one or more copies of a messenger RNA that codes for the insulin molecule, the insulin receptor, the enzyme prohormone convertase one, the enzyme prohormone convertase two, the enzyme carboxypeptidase E. Depending upon the physical size of the messenger RNAs and the available space inside the modified Hepatitis C virus more than one type of messenger RNA may be packaged into a single modified Hepatitis C virus, which would produce more than one therapeutic action in a cell. The modified Hepatitis C virus would be incapable of replication on its own due to the fact that the messenger RNA that a naturally occurring Hepatitis C virus would normally carry would not be present in the modified form of the Hepatitis C virus.

To treat diabetes, a quantity of modified Hepatitis C virus would be introduced into a patient's blood stream or tissues so that the modified virus could deliver the therapeutic genetic payload that it carries to beta cells in the pancreas. When the probes on the surface of the modified Hepatitis C virus engage a cell-surface receptor or receptors on a beta cell, the modified Hepatitis C virus will insert its therapeutic payload of messenger RNA into the beta cell to enhance the beta cell's biologic function of producing insulin and/or insulin receptors.

By providing beta cells with the above-mentioned messenger RNAs, the capacity of beta cells to carrying out the biologic processes of producing insulin and recognizing and responding to blood glucose levels is enhanced, which results in an efficient means to control the glucose levels in the blood stream on a constant and persistent basis utilizing innate regulatory mechanisms and thus diabetes mellitus can be effectively treated and the harmful effects of this disease can be averted.

What is claimed:

1) A medical treatment device that consists of a modified form of a virus that contains one or more copies of a medically therapeutic messenger ribonucleic acid molecule.

2) A medical treatment device that consists of a modified form of a virus that contains one or more copies of a medically therapeutic messenger ribonucleic acid molecule for the purpose of inserting into a biologically active cell one or more messenger ribonucleic acid molecules that are capable of interacting with one or more ribosomes to produce a specific protein inside the said metabolically active cell for the purpose of carrying out a therapeutic medical treatment.

3) A medical treatment device that consists of a modified form of a virus carrying on its surface a quantity of probes that target cell-surface receptors on certain cells, this said modified virus containing one or more copies of a medically therapeutic messenger ribonucleic acid molecule for the purpose of inserting into a specific biologically active cell one or more messenger ribonucleic acid molecules that are capable of interacting with one or more ribosomes to produce a specific protein inside the said metabolically active cell for the purpose of carrying out a therapeutic medical treatment.

4) A medical treatment device that consists of a modified form of a virus that contains one or more copies of a medically therapeutic messenger ribonucleic acid molecule and the enzymes required to modify the molecule or molecules of the said messenger ribonucleic acid to create one or more biologically active subunits of the said messenger ribonucleic acid molecule.

5) A medical treatment device that consists of a modified form of a virus that contains one or more copies of a messenger ribonucleic acid molecule and the enzymes required to modify the molecule or molecules of said messenger ribonucleic acid to create one or more biologically active subunits of the said messenger ribonucleic acid molecule for the purpose of inserting into a biologically active cell one or more messenger ribonucleic acid molecules that are capable or will become capable following modification of said messenger ribonucleic acid molecule, of interacting with one or more ribosomes to produce a specific protein inside the said metabolically active cell.

6) A medical treatment device that consists of a modified form of a virus that contains one or more copies of a messenger ribonucleic acid molecule and the enzymes required to modify the molecule or molecules of said messenger ribonucleic acid to create one or more biologically active subunits of the said messenger ribonucleic acid molecule for the purpose of inserting into a biologically active cell one or more messenger ribonucleic acid molecules that are capable or will become capable following modification of said messenger ribonucleic acid molecule, of interacting with one or more ribosomes to produce a specific protein inside the said metabolically active cell for the purpose of carrying out a therapeutic medical treatment.

7) A medical treatment device that consists of a modified Hepatitis C virus, modified to have a quantity of surface probes to recognize and engage one or more exterior cell-surface receptors on the surface of a beta cell located in the Islets of Langerhans in the pancreas and the said medical device having a quantity of surface probes that will facilitate inserting the modified Hepatitis C virus's genome that it carries into the target beta cell, the Hepatitis C virus further modified to have its own innate positive sense ribonucleic acid genome replaced by one or more copies of the messenger ribonucleic acid that codes for the manufacture of the ‘pro-insulin’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘pro-insulin’ molecules.

8) A medical treatment device that consists of a modified Hepatitis C virus, modified to have a quantity of surface probes to recognize and engage one or more exterior cell-surface receptors on the surface of a beta cell located in the Islets of Langerhans in the pancreas and the said medical device having a quantity of surface probes that will facilitate inserting the modified Hepatitis C virus's genome that it carries into the target beta cell, the Hepatitis C virus further modified to have its own innate positive sense ribonucleic acid genome replaced by one or more copies of the messenger ribonucleic acid that codes for the manufacture of the ‘insulin receptor’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘insulin receptor’ molecules.

9) A medical treatment device that consists of a modified Hepatitis C virus, modified to have a quantity of surface probes to recognize and engage one or more exterior cell-surface receptors on the surface of a beta cell located in the Islets of Langerhans in the pancreas and the said medical device having a quantity of surface probes that will facilitate inserting the modified Hepatitis C virus's genome that it carries into the target beta cell, the Hepatitis C virus further modified to have its own innate positive sense ribonucleic acid genome replaced by one or more copies of the messenger ribonucleic acid that codes for the manufacture of the ‘prohormone convertase one (PC1)’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘prohormone convertase one (PC1)’ molecules.

10) A medical treatment device that consists of a modified Hepatitis C virus, modified to have a quantity of surface probes to recognize and engage one or more exterior cell-surface receptors on the surface of a beta cell located in the Islets of Langerhans in the pancreas and the said medical device having a quantity of surface probes that will facilitate inserting the modified Hepatitis C virus's genome that it carries into the target beta cell, the Hepatitis C virus further modified to have its own innate positive sense ribonucleic acid genome replaced by one or more copies of the messenger ribonucleic acid that codes for the manufacture of the ‘prohormone convertase two (PC2)’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘prohormone convertase two (PC2)’ molecules.

11) A medical treatment device that consists of a modified Hepatitis C virus, modified to have a quantity of surface probes to recognize and engage one or more exterior cell-surface receptors on the surface of a beta cell located in the Islets of Langerhans in the pancreas and the said medical device having a quantity of surface probes that will facilitate inserting the modified Hepatitis C virus's genome that it carries into the target beta cell, the Hepatitis C virus further modified to have its own innate positive sense ribonucleic acid genome replaced by one or more copies of the messenger ribonucleic acid that codes for the manufacture of the ‘carboxypeptidase E’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘carboxypeptidase E’ molecules.

12) A medical treatment device that consists of a modified Hepatitis C virus, modified to have a quantity of surface probes to recognize and engage one or more exterior cell-surface receptors on the surface of a beta cell located in the Islets of Langerhans in the pancreas and the said medical device having a quantity of surface probes that will facilitate inserting the modified Hepatitis C virus's genome that it carries into the target beta cell, the Hepatitis C virus further modified to have its own innate positive sense ribonucleic acid genome replaced by one or more copies of the messenger ribonucleic acid that codes for the manufacture of the ‘insulin’ molecule for the purpose of inserting into a beta cell one or more copies of the said messenger ribonucleic acid molecule with the objective being the ribosomes in the beta cell are utilize one or more of the said messenger ribonucleic acid molecules to manufacture ‘insulin’ molecules.

13) A medical treatment device that consists of a modified Hepatitis C virus, modified to have a quantity of surface probes to recognize and functionally engage one or more exterior cell-surface receptors on the surface of a beta cell located in the Islets of Langerhans in the pancreas and the said medical device having a quantity of surface probes that will facilitate inserting the modified Hepatitis C virus's genome that it carries into the target beta cell, the Hepatitis C virus further modified to have its own innate positive sense ribonucleic acid genome replaced by one or more copies of messenger ribonucleic acids that code for the manufacture of two or more of any of the proteins including ‘pro-insulin, insulin, insulin receptor, prohormone convertase one (PC1), prohormone convertase two (PC2), carboxypeptidase E’ for the purpose of inserting into a beta cell one or more copies of the said messenger ribonucleic acid molecules with the objective being the ribosomes in the beta cell are to utilize two or more of the said messenger ribonucleic acid molecules to manufacture two or more proteins including ‘pro-insulin, insulin, insulin receptor, prohormone convertase one (PC1), prohormone convertase two (PC2), and/or carboxypeptidase E’ as necessary to successfully and effectively manage elevated blood glucose levels.

14) A medical treatment device that consists of a modified Hepatitis C virus, modified to have a quantity of surface probes to recognize and functionally engage one or more GPR40 exterior cell-surface receptors present on the surface of beta cells located in the Islets of Langerhans in the pancreas and the said medical device having a quantity of surface probes that will facilitate inserting the modified Hepatitis C virus's genome that it carries into a target beta cell once the GPR40 cell-surface receptor has been properly engaged, the Hepatitis C virus further modified to have its own innate positive sense ribonucleic acid genome replaced by one or more copies of the messenger ribonucleic acid that codes for the manufacture of the ‘pro-insulin’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘pro-insulin’ molecules.

15) A medical treatment device that consists of a modified Hepatitis C virus, modified to have a quantity of surface probes to recognize and functionally engage one or more GPR40 exterior cell-surface receptors present on the surface of beta cells located in the Islets of Langerhans in the pancreas and the said medical device having a quantity of surface probes that will facilitate inserting the modified Hepatitis C virus's genome that it carries into a target beta cell once the GPR40 cell-surface receptor has been properly engaged, the Hepatitis C virus further modified to have its own innate positive sense ribonucleic acid genome replaced by one or more copies of the messenger ribonucleic acid that codes for the manufacture of the ‘insulin receptor’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘insulin receptor’ molecules.

16) A medical treatment device that consists of a modified Hepatitis C virus, modified to have a quantity of surface probes to recognize and functionally engage one or more GPR40 exterior cell-surface receptors present on the surface of beta cells located in the Islets of Langerhans in the pancreas and the said medical device having a quantity of surface probes that will facilitate inserting the modified Hepatitis C virus's genome that it carries into a target beta cell once the GPR40 cell-surface receptor has been properly engaged, the Hepatitis C virus further modified to have its own innate positive sense ribonucleic acid genome replaced by one or more copies of the messenger ribonucleic acid that codes for the manufacture of the ‘prohormone convertase one (PC1)’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘prohormone convertase one (PC1)’ molecules.

17) A medical treatment device that consists of a modified Hepatitis C virus, modified to have a quantity of surface probes to recognize and functionally engage one or more GPR40 exterior cell-surface receptors present on the surface of beta cells located in the Islets of Langerhans in the pancreas and the said medical device having a quantity of surface probes that will facilitate inserting the modified Hepatitis C virus's genome that it carries into a target beta cell once the GPR40 cell-surface receptor has been properly engaged, the Hepatitis C virus further modified to have its own innate positive sense ribonucleic acid genome replaced by one or more copies of the messenger ribonucleic acid that codes for the manufacture of the ‘pro-insulin’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘prohormone convertase two (PC2)’ molecules.

18) A medical treatment device that consists of a modified Hepatitis C virus, modified to have a quantity of surface probes to recognize and functionally engage one or more GPR40 exterior cell-surface receptors present on the surface of beta cells located in the Islets of Langerhans in the pancreas and the said medical device having a quantity of surface probes that will facilitate inserting the modified Hepatitis C virus's genome that it carries into a target beta cell once the GPR40 cell-surface receptor has been properly engaged, the Hepatitis C virus further modified to have its own innate positive sense ribonucleic acid genome replaced by one or more copies of the messenger ribonucleic acid that codes for the manufacture of the ‘carboxypeptidase E’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘carboxypeptidase E’ molecules.

19) A medical treatment device that consists of a modified Hepatitis C virus, modified to have a quantity of surface probes to recognize and functionally engage one or more GPR40 exterior cell-surface receptors present on the surface of beta cells located in the Islets of Langerhans in the pancreas and the said medical device having a quantity of surface probes that will facilitate inserting the modified Hepatitis C virus's genome that it carries into a target beta cell once the GPR40 cell-surface receptor has been properly engaged, the Hepatitis C virus further modified to have its own innate positive sense ribonucleic acid genome replaced by one or more copies of the messenger ribonucleic acid that codes for the manufacture of the ‘insulin’ molecule for the purpose of inserting into a beta cell one or more copies of the said messenger ribonucleic acid molecule with the objective being the ribosomes in the beta cell are to utilize one or more of the said messenger ribonucleic acid molecules to manufacture ‘insulin’ molecules.

20) A medical treatment device that consists of a modified Hepatitis C virus, modified to have a quantity of surface probes to recognize and functionally engage one or more GPR40 exterior cell-surface receptors present on the surface of beta cells located in the Islets of Langerhans in the pancreas and the said medical device having a quantity of surface probes that will facilitate inserting the modified Hepatitis C virus's genome that it carries into a target beta cell once the GPR40 cell-surface receptor has been properly engaged, the Hepatitis C virus further modified to have its own innate positive sense ribonucleic acid genome replaced by one or more copies of messenger ribonucleic acids that code for the manufacture of two or more of the following proteins including ‘pro-insulin, insulin, insulin receptor, prohormone convertase one (PC1), prohormone convertase two (PC2), carboxypeptidase E’ for the purpose of inserting into a beta cell one or more copies of the said messenger ribonucleic acid molecules with the objective being the ribosomes in the beta cell are utilize two or more of the said messenger ribonucleic acid molecules to manufacture two or more proteins including ‘pro-insulin, insulin, insulin receptor, prohormone convertase one (PC1), prohormone convertase two (PC2), and/or carboxypeptidase E’ as necessary to successfully and effectively manage elevated blood glucose levels.
